(19) United States
(12) Patent Application Publication
(10) Pub. No.: US 2013/0012869 A1
(43) Pub. Date: Jan. 10, 2013

(54) NASAL IRRIGATION SYSTEMS

(76) Inventors: Albert CHA, Los Altos, CA (US); John SPIRIDIGLIOZZI, San Mateo, CA (US); Ralph NIVEN, Half Moon Bay, CA (US)

(21) Appl. No.: 13/545,859

(22) Filed: Jul. 10, 2012

Related U.S. Application Data
(63) Continuation of application No. PCT/US2011/023796, filed on Feb. 4, 2011.
(60) Provisional application No. 61/303,147, filed on Feb. 10, 2010.

(51) Int. Cl. A61M 3/02

(52) U.S. Cl. 604/28; 604/30

(57) ABSTRACT

Nasal irrigation systems for irrigating and/or rinsing a subject's nasal cavity are disclosed herein. Irrigation fluid may be drawn through the nasal cavity while utilizing a staged treatment procedure which allows for an initial infusion or flushing of irrigation fluid, circulation of the fluid, and subsequent flushing of the effluent from the nasal cavity. The system allows for reversible flow of the fluid during irrigation as well as use of vibration to disrupt debris within the nasal cavity to facilitate the mixing and removal of the debris. Additionally, peristaltic flow of the irrigation fluid may be used to facilitate contact between the fluid and debris during fluid circulation. Moreover, the irrigation fluid may also incorporate air or a gas into the fluid flow to create discrete volumes or boluses of pressured fluid to further facilitate thorough irrigation of the nasal cavity.

### CROSS-REFERENCE TO RELATED APPLICATIONS

This application is a continuation of International Patent Application No. PCT/US2011/023796 filed Feb. 4, 2011 which claims the benefit of U.S. Provisional Patent Application No. 61/303,147 filed on Feb. 10, 2010, the contents of which are incorporated herein by reference in their entirety.

### FIELD OF THE INVENTION

The present invention relates to methods and apparatus for irrigating and/or rinsing a subject's nasal cavity. More particularly, the present invention relates to methods and apparatus for irrigating and/or rinsing a subject's nasal cavity by utilizing various features to facilitate such procedures.

### BACKGROUND OF THE INVENTION

Regular implementation of nasal irrigation and/of rinsing of a subject's nasal cavity are generally an effective therapy to relieve symptoms associated with nasal problems such as rhinosinusitis, upper respiratory infections, allergies, etc. Conventional treatments for effecting nasal irrigation have generally included gravity-based and pressure-based devices.

Typical gravity-based devices flow saline into the nasal cavity from a reservoir, such as a Neti pot, into one nostril and out the other nostril while flowing the fluid into the nasal cavity and through the nasal septum. Because these devices utilize gravity to effectuate fluid flow through the nasal cavity, the user must position themselves into an awkward position and must also perform the procedure in an area where the irrigated fluid may be captured or trapped such as in a sink or large container.

Other gravity-based systems have attempted to facilitate nasal irrigation by utilizing hand-held devices which irrigate the nasal cavity under gravity and some suction to draw the fluid through the cavity while collecting the effluent material in a separate capture reservoir. However, such devices require the use of a relatively large irrigation reservoir to hold a sufficient volume of fluid and also require an equally large capture reservoir for containing the effluent material. The resulting device is large and bulky for the user to handle.

Alternative pressure-based devices typically utilize a pump to force saline fluid through the nasal cavity by introducing the fluid into one nostril and out the other nostril. Yet like gravity-based devices, the pressure-based devices require the use of a large volume of irrigation fluid as well as a large capture vessel to contain the effluent material and results in a bulky device.

Moreover, both gravity-based and pressure-based systems irrigate the nasal passages by simply flowing the irrigation fluid uni-directionally through the nasal cavity. One difficulty in effectively treating the nasal cavity is ensuring that all regions of the cavity have been suitably treated as a simple uni-directional flow may not result in complete irrigation of all tissue surfaces, thus simple irrigation through the cavity may not be entirely effective.

Accordingly, there exists a need for devices and methods which are effective yet easy to handle and use for irrigating and /or rinsing the nasal cavity of a subject.

### SUMMARY OF THE INVENTION

Irrigating and/or rinsing a subject's nasal cavity may be accomplished by utilizing a fluid reservoir which holds a volume of irrigating fluid, such as saline, as well as a capture reservoir for storing the effluent material. The irrigation fluid may be introduced into one nostril and drawn through the nasal cavity, across the nasal septum via the posterior margin and out the other nostril. Although irrigation and/or rinsing of the nasal cavity in particular are described, other bodily cavities may be treated utilizing the devices and methods described herein such as the paranasal cavities, e.g., maxillary sinuses, frontal sinuses, sphenoid sinuses, nasopharynx, etc. A staged treatment procedure which allows for an initial infusion or flushing of irrigation fluid, circulation of the fluid, and subsequent flushing of the effluent from a subject's nasal cavity may be utilized. Other features may incorporate a reversible flow of the irrigation fluid during an irrigation procedure as well as the use of vibration to potentially disrupt debris within the nasal cavity to facilitate the mixing and removal of the debris with the irrigated fluid for removal from the cavity. Additional features may incorporate the use of pulsed fluid flow, e.g., via a peristaltic flow of the irrigation fluid, to facilitate contact between the fluid and debris during fluid circulation, as described in further detail below. Alternatively and/or additionally, the irrigation fluid may also incorporate air or a gas into the fluid flow to create discrete volumes or boluses of pressured fluid to further facilitate thorough irrigation of the nasal cavity.

One variation of a nasal irrigation assembly may be fluidly coupled to a fluid reservoir which may hold a volume of irrigating fluid coupled to the fluid channel. A fluid actuation mechanism, e.g., a fluid pump (such as a reversible peristaltic pump) which is manually or automatically operable, may be integrated with the fluid reservoir and actuatable to urge or force the irrigating fluid from the reservoir and into the fluid channel. While the fluid reservoir may be sized to accommodate any range of irrigating fluid volumes, the reservoir may be sized to hold, e.g.,3 to 20 cc or more of the irrigation fluid. Moreover, the irrigation fluid itself may comprise saline fluid optionally infused with one or more drugs or agents, e.g., steroids, vaso-constrictors, etc. for administering additional treatments to the nasal cavity tissues as well as mild surfactants to break up mucus during irrigation and to help clear nasal passages. Other fluids aside from saline may be utilized as well. Furthermore, the irrigation fluid may also range in concentration to be, e.g., isotonic, hypotonic, hypertonic, etc., as so desired.

A capture reservoir may also be fluidly coupled to the fluid channel for receiving the effluent material during irrigation. An operable valve, e.g., stopcock, may. also be in communication between the reservoir and fluid channel to selectively direct flow either to the reservoir or to circulate through the fluid channel. Additionally, a valve, e.g., uni-directional valve, may also be incorporated along the fluid channel to direct the irrigation fluid flow in a single direction. An additional filter may also be incorporated along the fluid channel on either side of the valve to filter and capture any debris which may be circulating through the fluid channel during fluid irrigation or circulation. Optionally, a heating element may also be integrated into any of the components to warm the irrigation fluid.

With the valve suitably actuated, an initial flush of sterile irrigation fluid may be pumped from the reservoir into the fluid channel and through the first lumen opening for introduction into the subject's nasal cavity to purge the device and nasal cavity of air as well as any large debris and/or viscous mucous from the cavity. The irrigated fluid received from the subject's nostril may pass into the second lumen opening, partly through the fluid channel, and into the capture reservoir such that any large debris and/or viscous mucous may be contained. Additionally, any trapped air or gas may be vented from the nasal cavity, device, and/or the capture reservoir through the vent defined in the reservoir. A relatively small volume of the irrigated fluid, e.g., 3 to 10 cc or more, may be directed via the valve to flow into the capture reservoir for this initial purge. Optionally, an irrigation fluid having a viscosity altered from the viscosity of saline (relatively higher or lower) may be used for the initial pass, e.g., ethanol alcohol solution mixed with saline, glycerin, propylene glycol, etc., to facilitate the clearing of debris and/or mucous as well as to facilitate any deposition of drugs which may be infused with the irrigation fluid. Subsequent irrigation cycles may utilize a fluid having a relatively lower viscosity, if so desired.

After the initial purge, the remaining volume of irrigation fluid within the fluid reservoir, e.g., the remaining 10 to 20 cc or more, may be then introduced into the fluid channel for introduction into and through the nasal cavity. The valve may be actuated to allow flow through the fluid channel while restricting flow into the capture reservoir such that the irrigation fluid cycled through the nasal cavity may be recirculated through the device and back into the nasal cavity to ensure thorough irrigation and/or rinsing. A pumping mechanism may urge or drive the recirculating fluid through the fluid channel and the nasal cavity. A uni-directional valve may ensure that the recirculating fluid flows in a single direction while a filter may capture any debris dislodged from the nasal cavity during the recirculatory flow to ensure that the dislodged debris is prevented from flowing back into the nasal cavity. The filtered fluid may be recirculated through the nasal cavity for one or more passes, e.g. two passes, to thoroughly irrigate and rinse the tissue. Because the irrigation fluid is recirculated, the total volume of fluid needed to effectively irrigate and/or rinse the nasal cavity is greatly reduced from a typical gravity or pressure-based design and allows for the assembly to have a relatively compact form factor for ease of handling.

Once the irrigation fluid has been introduced and recirculated, the valve may be actuated to re-direct the flow from the fluid channel back into the capture reservoir to cease the recirculation of fluid. The recirculating fluid may be accordingly drained to capture any remaining debris and/or mucous and a final purge of air or gas may be optionally introduced into the device and nasal cavity to purge any of the remaining fluid. To introduce the purging air or gas, the fluid reservoir, e.g., may be re-filled with air, or air may be introduced into the system through an alternative valve and this air or gas may then be introduced as a final purging step. Additionally and/or alternatively, along with (or in place of) the purging air or gas another fluid mixture may be introduced. For instance, a fluid mixture containing, e.g., hydrogen peroxide (H₂O₂), ethanol mixture, or other sterilizing agent, etc., may be introduced during or after fluid circulation to sterilize and to completely purge the nasal cavity as well as the device.

In another variation rather than utilizing a positive-pressure pumping mechanism, a negative-pressure system may be used. An aspiration chamber having the fluid actuation mechanism may be fluidly coupled through the valve to the fluid channel and a fluid reservoir may likewise be fluidly coupled through the valve to the fluid channel.

With the respective valves set in a purging position, the aspiration chamber may be actuated to draw the irrigation fluid from the fluid reservoir through the fluid channel, through the nasal cavity, and directly into the aspiration chamber for an initial purge to remove any large debris and also to purge the system and nasal passages of air. The valves may be set to enable flow through only the fluid channel and the aspiration chamber may be optionally removed. With the pump actuated forcing fluid flow through a bypass lumen and the valves set to allow flow through the fluid channel, the irrigation fluid may be recirculated through the fluid channel, bypass lumen, and into the subject's nasal cavity through the lumen opening and back. One or more filters may be incorporated along the fluid channel to capture any debris and prevent its recirculation through the nasal cavity. Once the recirculating fluid has passed through the nasal cavity for at least one or two (or more) passes, the valves may be reset to a purging position to redirect the fluid flow into the aspiration chamber until all remaining fluid from the system and nasal cavity has been aspirated.

In another variation, a selector control may be actuated either manually by the user or automatically by a controller (such as a processor) integrated into the assembly to control the unidirectional purging flow, recirculating flow through the nasal cavity, and optional final purging step.

In yet another variation, any of the devices described herein may include a mask for temporary placement upon the user's face in proximity to their nose during a treatment to form a seal against the subject's nose and face to capture any fluid leakage which may occur from the nostril-port interface. The mask may incorporate one or more vibrating elements integrated along the mask or assembly which when engaged may vibrate the mask or a portion thereof to transmit vibrations to the underlying tissue or bones, such as the cheek bones, of the subject. These transmitted vibrations may be imparted to disturb any fluids which may be contained within the nasal cavity to cause any debris, such as hardened or thickened mucous, to dislodge from the sinus walls and to mix with the circulating irrigation fluid for flushing out of the nostril and into the capture reservoir. In other variations, a vibrational mechanism may be used to directly transmit vibrations through the irrigation fluid being circulated through the nasal cavity. Such vibrations can comprise a high frequency vibration such as ultrasonic vibrations or even low frequency vibrations, e.g., at a frequency of less than 1000 Hz, to cause the mucous to break down and drain more easily with the circulating irrigation fluid.

In yet another variation, a high-energy radiation source (e.g., having wavelengths between 185 nm to 245 nm such as an ultraviolet light source) may be optionally integrated into the irrigation assembly and positioned within the housing in proximity to the fluid channel such that the light source may irradiate the adjacent fluid channel and the irrigation fluid flowing therethrough.

Additionally and/or alternatively, any of the variations described herein may further comprise an optional valving feature to mix the irrigation fluid with air or a gas introduced into the fluid flow to form discrete volumes for flushing through the device and nasal cavity. This mixture of air (or gas) with irrigation fluid may allow for better disruption of debris within the nasal cavity.

### BRIEF DESCRIPTION OF THE DRAWINGS

FIG. 1 illustrates an example of a system which irrigates and/or rinses a subject's nasal cavity by utilizing a multi-stage circulatory process.

FIG. 2 illustrates an example of a hand-held device which may be used to treat the subject's nasal cavity.

FIGS. 3A to 3C illustrate one variation where irrigation fluid may be introduced into the nasal cavity to initially flush the nasal cavity, then recirculated to effectuate thorough irrigation, and then cleared from the nasal cavity.

FIGS. 3D to 3G illustrate another variation where irrigation fluid may be introduced into the nasal cavity, then recirculated to effectuate thorough irrigation, then reintroduced back into the same reservoir for removal.

FIGS. 4A and 4B illustrate another variation where the irrigation fluid may be drawn through the nasal cavity via negative pressure, such as a vacuum, to initially flush the nasal cavity.

FIGS. 4C and 4D illustrate where the irrigation fluid may be recirculated through the nasal cavity via a pumping mechanism, such as a peristaltic pump.

FIGS. 4E and 4F illustrate where the recirculated fluid may be subsequently drawn from the nasal cavity.

FIG. 5 shows another variation of a nasal irrigation system utilizing an irrigation fluid cartridge which is removably connected to an actuatable valve.

FIGS. 6A to 6D show respective side and top views of a selector control which may be actuated to effect a different stage during the irrigation process.

FIG. 7 shows a perspective view of another variation which may utilize one or more vibrating elements integrated into the device to effectuate removal of debris from the nasal cavity during irrigation.

FIG. 8 shows a front view of a device having the one or more vibrating elements integrated therein and an example of placement against the subject.

FIGS. 9A to 9C illustrate another example of a device utilizing a peristaltic pump to effect pulsed irrigation through multiple stages.

FIGS. 10A and 10B show an example where the peristaltic pump may be reversed in direction to effectuate reversed circulatory flow during a single procedure.

FIG. 11 shows another variation where an irradiating feature such as an ultraviolet light may be integrated in the device to sterilize the irrigation fluid.

FIG. 12A shows another variation where the device may incorporate a valve for drawing air or gas into the irrigation flow for effectuating a pulsed fluid flow through the nasal cavity.

FIG. 12B shows another variation where the device may incorporate a flapper valve for drawing air or gas into the fluid flow.

FIG. 12C shows yet another variation where the valve may be configured to open at a predetermined pressure to entrain air or gas into the fluid flow.

FIGS. 13A and 13B show side views of examples of traps which may be incorporated into the device for dampening fluid pulses as well as for capturing debris.

FIGS. 14A and 14B show side and front views of examples of distributor elements which may be rotatably positioned adjacent to the filter to clear the filter of collected debris and/or mucus.

FIG. 15 shows cross-sectional side and end views of another filtering assembly which may utilize a cartridge filter and trap assembly.

FIG. 16 shows side and front views of another variation of a filtering assembly which utilizes a rotatable blade or scraper element adjacent to the filter.

### DETAILED DESCRIPTION OF THE INVENTION

In irrigating and/or rinsing a subject's nasal cavity, any one of several features may be utilized individually and/or in combination to effectuate a thorough irrigation treatment. Generally, one variation of the device may incorporate a fluid reservoir which holds a volume of irrigating fluid, such as saline, as well as a capture reservoir for storing or capturing the effluent material. The irrigation fluid may be introduced into one nostril and drawn through the nasal cavity and out the other nostril. One feature may incorporate a staged treatment procedure which allows for an initial infusion or flushing of irrigation fluid, circulation of the fluid, and subsequent flushing of the effluent from a subject's nasal cavity. Other features may incorporate a reversible flow of the irrigation fluid during an irrigation procedure as well as the use of vibration to potentially disrupt debris within the nasal cavity to facilitate the mixing and removal of the debris with the irrigated fluid for removal from the cavity. Additional features may incorporate the use of a peristaltic flow of the irrigation fluid to facilitate contact between the fluid and debris during fluid circulation to disrupt the debris from adhering to the sinus cavity walls and to mix with the circulating fluid to increase the likelihood that it will be removed from the sinus cavity. Alternatively and/or additionally, the irrigation fluid may also incorporate air or a uas into the fluid flow to create discrete volumes or boluses of pressured fluid to further facilitate thorough irrigation of the nasal cavity.

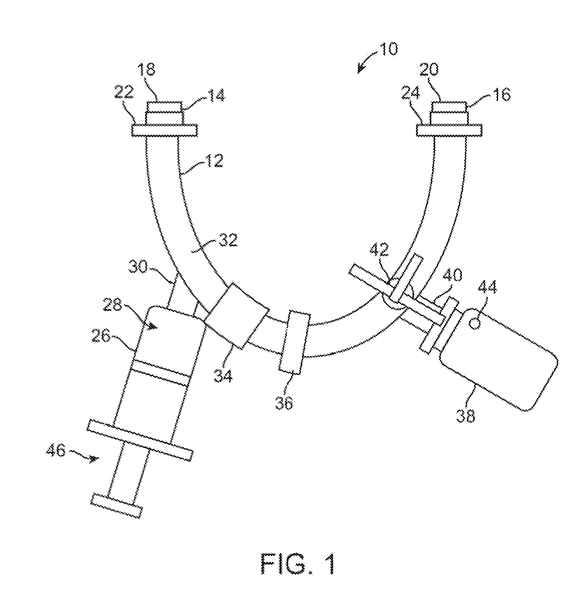

Turning now to the example shown in FIG. 1, one variation of a nasal irrigation assembly 10 is shown as having a fluid channel 12, e.g., tubing, defining lumen 32 therethrough which fluidly couples a first nasal port 14 which defines a first lumen opening 18 to a second nasal port 16 which defines a second lumen opening 20. Each of the nasal ports 14, 16 may be shaped and sized for insertion at least partially into or in proximity to a nostril of a subject. For example, first nasal port 14 may be introduced into or in proximity to a left nostril while the second nasal port 16 may be introduced into or in proximity to a right nostril of the subject when in use. Each of the nasal ports 14, 16 may further comprise a respective nostril seal 22, 24 which may be comprised of a conformable material, such as silicone, and are each configured or sized to form a fluid-tight seal about the nostril openings to allow pressure to build as well as to prevent fluid from leaking. Each of the nasal ports 14, 16 may also be configured to be uniform in size or shape while in other variations at least one of the nasal ports serving as an inlet port for introducing the irrigation fluid may configured to be relatively more conformable than the other nasal port which may serve as an outlet port for enhancing comfort. Alternatively or additionally, the inlet port may define a lumen opening which is relatively larger than the lumen opening of the outlet port to facilitate circulation of the irrigation fluid therethrough.

The fluid channel 12 may also be fluidly coupled to a fluid reservoir 26 which may hold a volume of irrigating fluid 28 coupled to fluid channel 12 via a fluid connection 30. A fluid actuation mechanism 46, e.g., a fluid pump which is manually or automatically operable, may be integrated with the fluid reservoir 26 and actuatable to urge or force the irrigating fluid 28 from the reservoir 26 and into fluid channel 12. Although fluid reservoir 26 and mechanism 46 are illustrated as a syringe in this variation, this is merely illustrative of a fluid reservoir which may be pressurized and any number of variations is intended to be included in this disclosure. While the fluid reservoir 26 may be sized to accommodate any range of irrigating fluid volumes, reservoir 26 may be sized in one example to hold, e.g., 3 to 20 cc or more of the irrigation fluid. Moreover, the irrigation fluid itself may comprise saline fluid optionally infused with one or more drugs or agents, e.g., steroids, vaso-constrictors, etc. for administering additional treatments to the nasal cavity tissues as well as mild surfactants to break up mucus during irrigation and to help clear nasal passages. Aside from saline, other fluids may be utilized as well. Furthermore, the irrigation fluid may also range in concentration to be, e.g., isotonic, hypotonic, hypertonic, etc., as so desired. Additionally and/or alternatively, fluids having an altered pH level from that of saline may also be utilized. For example, irrigation fluids having a relatively higher or lower pH level may be utilized in temporarily or permanently inactivating inflammatory proteases.

Aside from the fluid reservoir 26, a capture reservoir 38 may also be fluidly coupled to fluid channel 12 via a fluid connection 40 and may further include an optional vent 44 for displacing any gas or air within the capture reservoir 38 when receiving the effluent material during initial purging and final flushing stages. An operable valve 42, e.g., stopcock, may also be in communication between reservoir 38 and fluid channel 12 to selectively direct flow either to reservoir 38 or to circulate through fluid channel 12. Additionally, a valve 34, e.g., uni-directional valve, may also be incorporated along fluid channel 12 to direct the irrigation fluid flow in a single direction, particularly during recirculation as described in further detail below. An additional filter 36 may also be incorporated along fluid channel 12 on either side of valve 34 to filter and capture any debris which may be circulating through fluid channel 12 during fluid irrigation or circulation. Optionally, a heating element may also be integrated into any of the components to warm the irrigation fluid. For example, a heating element may be incorporated into fluid reservoir 26, connector 30, fluid channel 12, etc. so long as the heating element is in thermal communication with the irrigation fluid.

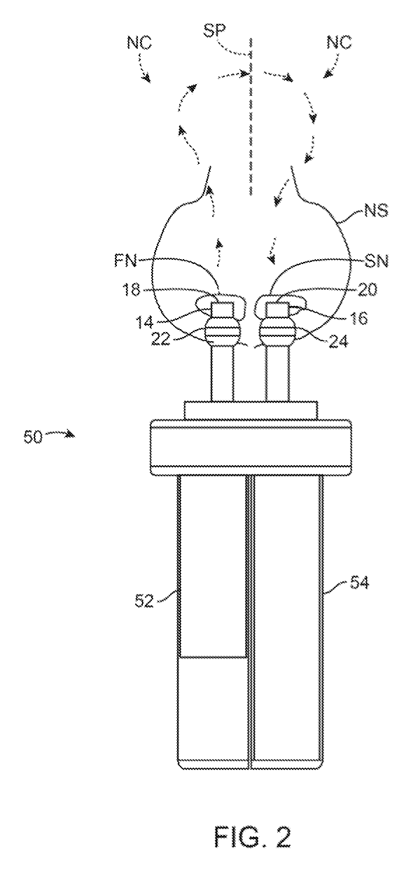

FIG. 2 illustrates an example of a variation for housing the various components into a compact nasal irrigation assembly 50 which is easily handled and manipulated by the user. As shown, housing 52 may incorporate the actuation mechanisms such as a pump, electronics, etc. as well as the fluid reservoir 26. Additionally cartridge 54 for housing the capture reservoir 38 may also be incorporated and optionally removable from assembly 50. Each of the nasal ports 14, 16 and respective seals 22, 24 may project from assembly 50 and may be positioned for insertion at least partially into or in proximity to a first nostril FN and second nostril SN when held or positioned beneath the subject's nose NS. As previously described, each of the nasal ports 14, 16 and respective seals 22, 24 may temporarily form a fluid-tight seal between each respective nostril FN, SN. In this and other examples, the irrigation fluid introduced into, e.g., the first nostril FS, from first lumen opening 18 may flow through the nasal cavity NC, through the posterior margin, and into the adjacent nasal cavity NC to exit through the second nostril SN and into second lumen opening 20.

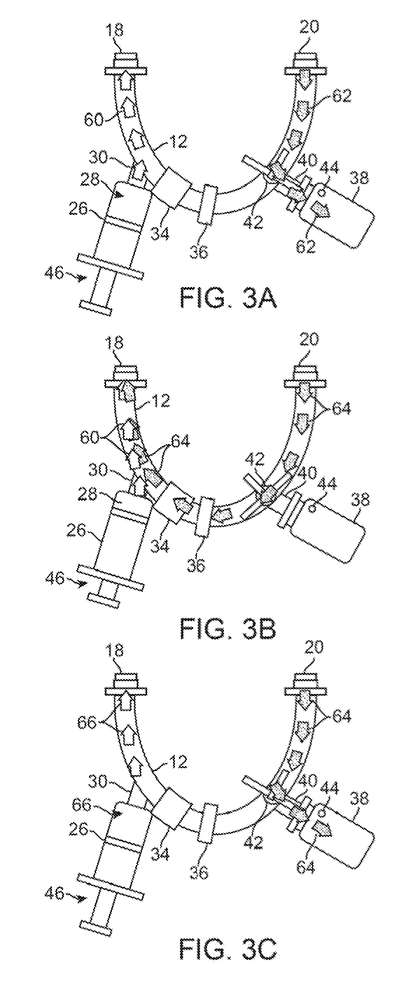

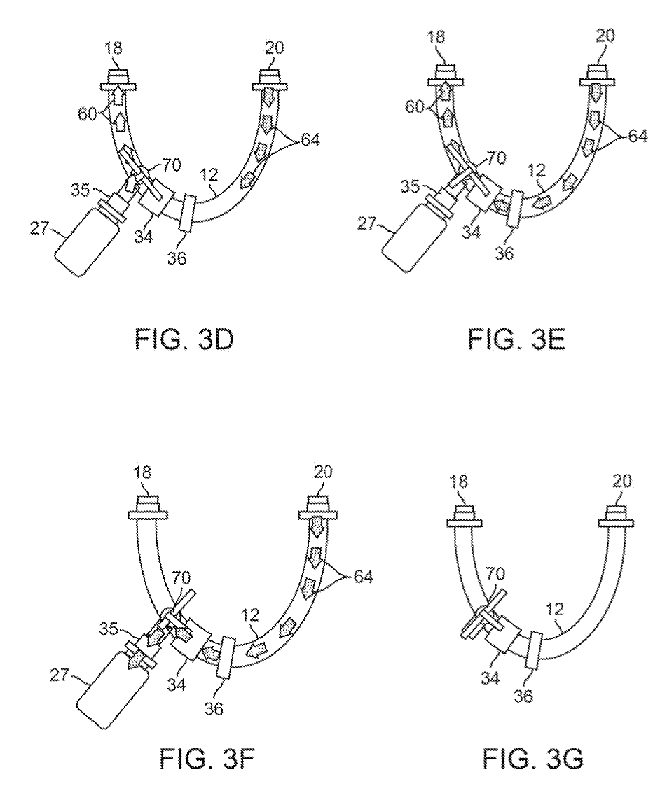

In use, an example of a staged procedure (e.g., a three-stage procedure) is shown illustratively in FIGS. 3A to 3C. With valve 42 suitably actuated, an initial flush of sterile irrigation fluid 60 may be pumped via mechanism 46 from reservoir 28 into fluid channel 12 and through first lumen opening 18 for introduction into the subject's nasal cavity to purge the device and nasal cavity of air as well as any large debris and/or viscous mucous from the cavity. The irrigated fluid 62 received from the subject's nostril may pass into second lumen opening 20, partly through fluid channel 12, and into capture reservoir 38 such that any large debris and/or viscous mucous may be captured within the capture reservoir 38. Additionally, any trapped air or gas may be vented from the nasal cavity, device, and/or capture reservoir 38 through vent 44 defined in reservoir 38. A relatively small volume of the irrigated fluid 62, e.g., 3 to 10 cc or more, may be directed via valve 42 to flow into capture reservoir 38 for this initial purge, as shown in FIG. 3A.

Optionally, an irrigation fluid having a relatively higher viscosity than saline may be used for the initial pass, e.g., ethanol alcohol solution mixed with saline, to facilitate the clearing of debris and/or mucous as well as to facilitate any deposition of drugs which may be infused with the irrigation fluid. Subsequent irrigation cycles may utilize a fluid having a relatively lower viscosity, if so desired.

After the initial purge, the remaining volume of irrigation fluid 60 within fluid reservoir 26, e.g., the remaining 10 to 20 cc or more, may be then introduced into fluid channel 12 for introduction into and through the nasal cavity. Valve 42 may be actuated to allow flow through fluid channel 12 while restricting flow into capture reservoir 38 such that the irrigation fluid cycled through the nasal cavity may be recirculated through the device and back into the nasal cavity to ensure thorough irrigation and/or rinsing, as shown in FIG. 3B. Pumping mechanism 46 may urge or drive the recirculating fluid 64 through fluid channel 12 and the nasal cavity. The uni-directional valve 34 may ensure that the recirculating fluid 64 flows in a single direction while filter'36 may capture any debris dislodged from the nasal cavity during the recirculatory flow to ensure that the dislodged debris is prevented from flowing back into the nasal cavity. The filtered fluid may be recirculated through the nasal cavity for one or more passes, e.g. two passes, to thoroughly irrigate and rinse the tissue. Because the irrigation fluid is recirculated, the total volume of fluid needed to effectively irrigate and/or rinse the nasal cavity is greatly reduced from a typical gravity or pressure-based design and allows for the assembly to have a relatively compact form factor for ease of handling.

Once the irrigation fluid 60 has been introduced and recirculated, valve 42 may be actuated to re-direct the flow from fluid channel 12 back into capture reservoir 38 to cease the recirculation of fluid. The recirculating fluid 64 may be accordingly drained to capture any remaining debris and/or mucous and a final purge of air or gas 66 may be optionally introduced into the device and nasal cavity to purge any of the remaining fluid, as shown in FIG. 3C. To introduce the purging air or gas 66, the fluid reservoir 26, e.g., may be re-filled with air, or air may be introduced into the system through an alternative valve (as described in further detail below) and this air or gas may then be introduced as a final purging step. Additionally and/or alternatively, along with (or in place of) the purging air or gas 66, another fluid mixture may be introduced. For instance, a fluid mixture containing, e.g., hydrogen peroxide (H₂O₂), ethanol mixture, or other sterilizing agent, etc., may be introduced during or after fluid circulation to sterilize and to completely purge the nasal cavity as well as the device.

Another variation which utilizes a single reservoir which may be used as both the fluid reservoir and capture reservoir is shown in the example of FIGS. 3D to 3G. In this particular variation, reservoir 27 may be initially filled with the irrigation fluid and attachable to fluid channel 12 as a removable cartridge. Because a single reservoir may be used to provide the irrigation fluid, which may be recirculated through the nasal cavity, and then used to capture the circulated fluid, the volume of irrigation fluid needed to effectively irrigate the nasal cavity is substantially reduced from conventional systems. The reservoir 27 may accordingly comprise a cartridge-like reservoir which may be removed from fluid channel 12 for facilitating disposal of the captured effluent material from reservoir 27 or for the disposal of the entire reservoir 27 itself. Cartridge reservoir 27 may then be cleaned and/or re-filled with additional irrigation fluid for re-use or another cartridge reservoir 27 may then be coupled to fluid channel 12 for subsequent use of the device, if so desired. The initial volume of irrigation fluid contained within the reservoir 27 may be substantially reduced from the volume needed in other conventional devices. Thus, a reservoir having a volume of irrigation fluid may ranee anywhere from, e.g., 3 cc to 20 cc, as previously described. For instance, the reservoir 27 may have predetermined volumes of irrigation fluid in various amounts, e.g., 20 cc or less, 15 cc or less, 10 cc or less, etc.

In use, reservoir 27 having the predetermined volume of irrigation fluid may be attached to fluid channel 12. Reservoir 27 may optionally incorporate a filter 35 such that the irrigation fluid 60 urged from reservoir 27 (e.g., via a pump integrated with reservoir 27 or coupled to reservoir 27 as described herein) is filtered and/or the recirculated fluid and/or effluent fluid when captured back in reservoir 27. As shown in FIG. 3D, valve 70 may be actuated to allow for the irrigation fluid 60 to pass from reservoir 27, through filter 35, and into fluid channel 12 where the irrigation fluid 60 may be urged into the nasal cavity, e.g., through lumen opening 18. The fluid passed through the nasal cavity may pass back into lumen opening 20 and into fluid channel 12 where the fluid may then be recirculated. Filter 36 may also be optionally incorporated into fluid channel 12 as well as another optional uni-directional valve 34 to ensure the recirculated fluid is flowed through the nasal cavity and fluid channel 12 in a single direction, as shown in FIG. 3E. In the event that the filter 35 is incorporated with the removable reservoir 27, filter 34 may be omitted from fluid channel 12. If filter 35 is incorporated with reservoir 27, they may be combined in a singular cartridge housing removably attached to the rest of the assembly. Alternatively, both the filter 35 in the removable cartridge and filter 34 may both be used if so desired.

Once the recirculated fluid 64 has been cycled through the device and nasal cavity, e.g., for two or more passes, valve 70 may be actuated again to direct the recirculated fluid 64 back into reservoir 27 which may have been partially or fully emptied of the initial irrigation fluid 60 and as shown in FIG. 3F. With the recirculated fluid 64 emptied from the nasal cavity and fluid channel 12 and back into reservoir 27, reservoir 27 along with the integrated filter 35 both may then be optionally removed from fluid channel 12 and emptied of the effluent material or disposed of entirely, as shown in FIG. 3G. A re-filled or new reservoir cartridge 27 and filter 35 (if optionally incorporated) may then be attached to the device for subsequent use.

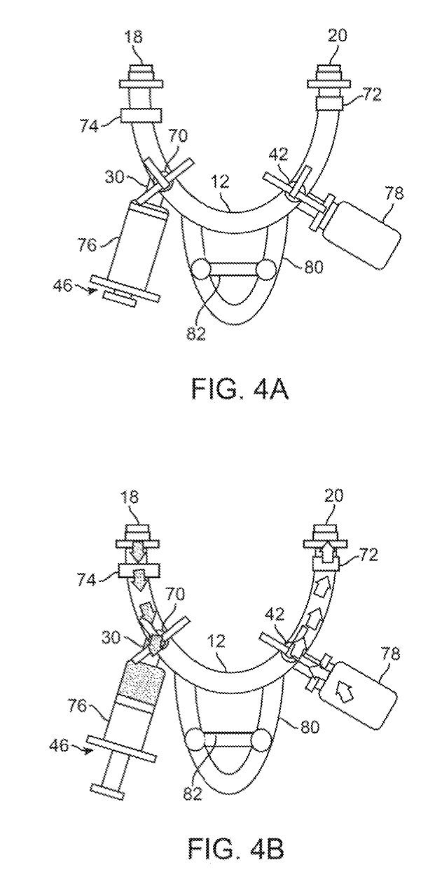

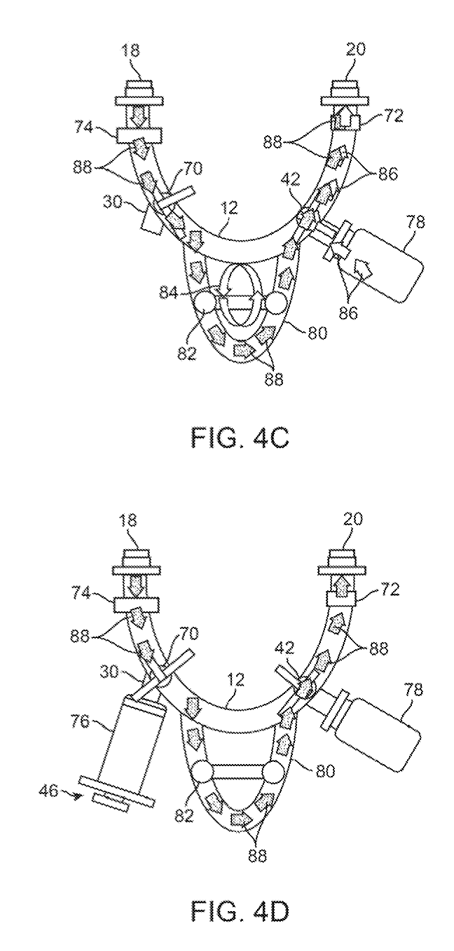

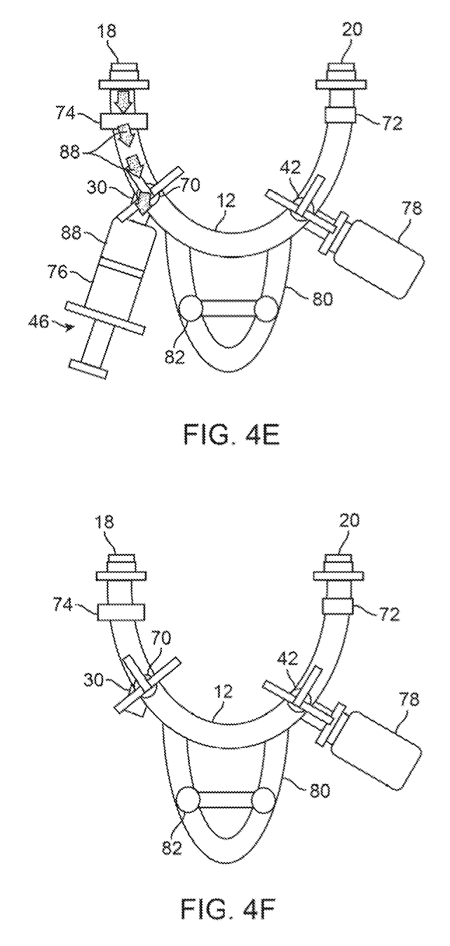

In another variation rather than utilizing a positive-pressure pumping mechanism, a negative-pressure system may be used, as shown in the example of FIGS. 4A and 4B. An aspiration chamber 76 having fluid actuation mechanism 46 may be fluidly coupled through valve 70 to fluid channel 12 and a fluid reservoir 78 may likewise be fluidly coupled through valve 42 to fluid channel 12, as shown in FIG. 4A and as previously described. As above, while aspiration chamber 76 and mechanism 46 is shown as a syringe for illustrative purposes, this is not intended to be limiting as other pumping mechanisms may be utilized. Additionally, stationary pumping mechanism typically found in clinical settings may also be utilized, if so desired. In this variation, an additional bypass lumen 80 coupled to fluid channel 12 and an additional pump 82, e.g., a reversible pump such as a peristaltic pump, in contact with bypass lumen 80 may also be incorporated into the device.

With respective valve 70 and 42 set in a purging position, aspiration chamber 76 may be actuated via mechanism 46 to draw the irrigation fluid from fluid reservoir 78 through fluid channel 12, through the nasal cavity, and directly into aspiration chamber 76 for an initial purge to remove any large debris and also to purge the system and nasal passages of air, as shown in FIG. 4B. As described above, an initial volume of irrigation fluid may be used for this initial purge. Valve 70 may be set to enable flow through only the fluid channel 12 and aspiration chamber 76 may be optionally removed. Valve 42 may also be set to enable flow through fluid channel 12 as well as from fluid reservoir 78 and pump 82 may be actuated in a first direction 84 (e.g., in a first direction of rotation in the case of a peristaltic pump such as the counter-clockwise flow direction shown) to urge the irrigation fluid 86 to flow through the fluid channel 12 and bypass lumen 80. Relief valve 72 positioned along fluid channel 12 may comprise a uni-directional valve to ensure that the fluid flows only in the desired direction and that the pressure stays within a predetermined level. With pump 82 actuated forcing fluid flow through the bypass lumen 80 and valves 42, 70 set to allow flow through fluid channel 12, the irrigation fluid 86 may be recirculated through the fluid channel 12, bypass lumen 80, and into the subject's nasal cavity through lumen opening 20 and back through opening 18, as shown in FIG. 4C. One or more filters 74 may be incorporated along fluid channel 12 to capture any debris and prevent its recirculation through the nasal cavity.

Once the recirculating fluid 88 has passed through the nasal cavity for at least one or two (or more) passes, valve 70 may be reset to a purging position to redirect the fluid flow into aspiration chamber 76, which may be re-attached to fluid channel 12 (if previously removed) prior to resetting valve 70 and prior to purging the fluid channel 12 and nasal cavity, as shown in FIG. 4D. With valve 70 redirecting the recirculating fluid 88, pump 82 may be optionally stopped and mechanism 46 may be actuated to create a negative pressure to draw the fluid 88 into chamber 76, as shown in FIG. 4E, until all remaining fluid from the system and nasal cavity has been aspirated into chamber 76. The aspiration chamber 76 may then removed from fluid channel 12, as shown in FIG. 4F, and emptied or discarded.

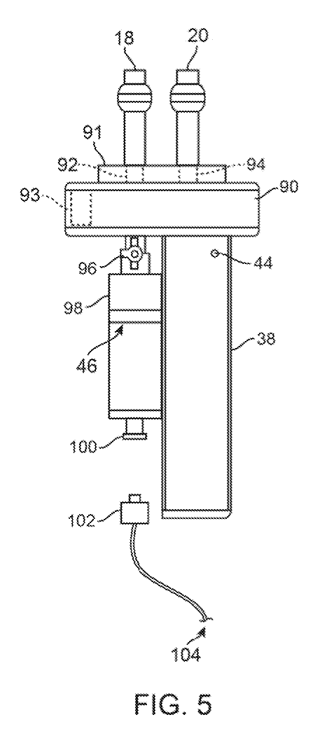

Another variation of a nasal irrigation and/or rinsing system is shown in the front view of FIG. 5 which illustrates another example of a device which may utilize a fluid reservoir containing the irrigation fluid, e.g., sterilized saline fluid, which is optionally detachable from the assembly. In this variation, (which shows part of the housing removed for clarity) the assembly may comprise a fluid reservoir 98 optionally configured as a container or cartridge which is removably detachable from an optional valve 96, e.g., a three-way valve or stopcock for allowing air to enter. Fluid reservoir 98 may also optionally comprise a port 100 which may be coupled to a port connector 102 in fluid communication with an opening 104 through which air or gas may enter. The valve 96 and capture reservoir 38, which may also be removably attached to the assembly, may be coupled to a selector control 90 which may be set in one of several positions to control the direction of fluid flow through the assembly. The assembly may further comprise a pump either integrated with the assembly, as previously described, or externally coupled. Moreover, an external pump may also be optionally utilized to supply pressurized air or gas delivered through opening 104 to urge the fluid flow through the device. The ports and lumen openings 18, 20 may be coupled to a stationary attachment 91 which is defines a respective first fluid lumen 92 and second fluid lumen 94 therethrough for attachment to the selector control 90.

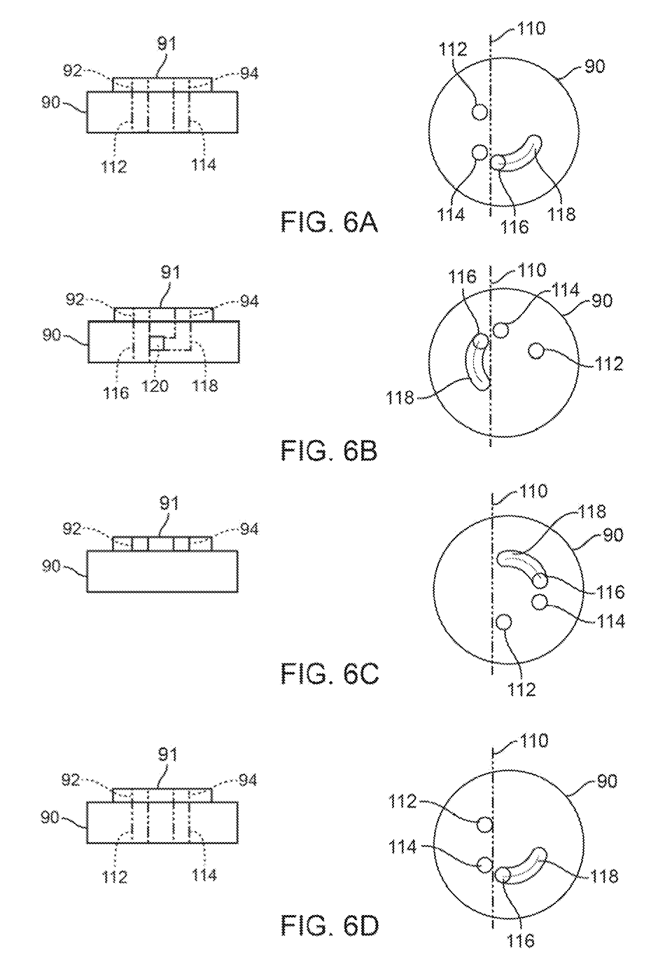

In use, the selector control 90 may be actuated either manually by the user or automatically by a controller (such as a processor 93) integrated into the assembly. As shown in the side and top views of FIG. 6A, a position of selector control 90 is shown relative to stationary attachment 91 and fluid lumens 92, 94 to illustrate how control 90 may be positioned to actuate different flow patterns depending upon the stage of irrigation or rinsing treatment. An indication line 110 is shown for explanatory purposes to delineate which fluid lumens defined through control 90 are actively engaged with the respective fluid lumens 92, 94. When control 90 is rotated to position first active lumen 112 and second active lumen 114 to the left of indication line 110 (with reference to FIG. 6A for explanatory purposes), each of the active lumens 112, 114 are in fluid communication with respective fluid lumens 92, 94 and unidirectional purging flow may occur where the irrigation fluid from fluid reservoir 98 may flow into the nasal cavity to initially flush debris and air, as described above.

Once the initial purging flow is completed, control 90 may be rotated, e.g., clockwise, relative to stationary attachment 91 to engage active lumen 116 into fluid communication with fluid lumen 92 and fluid reservoir 98 and to also engage bypass lumen 118 with fluid lumen 94 to allow for the recirculating flow through the nasal cavity, as shown in FIG. 6B and as also previously described. Bypass lumen 118 may be in fluid communication with active lumen 116 to allow for the recirculation of fluid through control 90 and it may also comprise valve 120 to ensure uni-directional fluid flow through the device and nasal cavity. The recirculating fluid flow may be actuated and/or maintained by pressurized air or gas introduced through fluid reservoir 98 or via a pump integrated with the device and as also previously described.

With completion of the recirculatory fluid flow treatment, control 90 may be actuated again by further rotating, e.g., clockwise, relative to stationary attachment 91 such that no active lumens are engaged with fluid lumens 92, 94, as indicated by the absence of any lumens relative to indication line 110 shown in FIG. 6C. In this position, the device may be optionally shut off and the fluid reservoir 98 may be removed, if desired, and optionally replaced by a supply of pressured air or gas. As an optional final step, with fluid reservoir 98 removed and replaced by a supply of air or gas (or with fluid reservoir 98 left in place), control 90 may be actuated again, e.g., clockwise, to re-engage active lumens 112, 114 with respective fluid lumens 92, 94 and air or gas may be introduced to purge the device and the nasal cavity of any remaining fluid, as shown in FIG. 6D.

Alternatively, rather than further actuating control 90 to the position shown in FIGS. 6C and 6D, valve 96 (shown in FIG. 5) may be actuated after recirculation is completed to allow for ambient air to enter into fluid lumens 92, 94 to purge the device and nasal cavity of any remaining irrigation fluid. In this manner, fluid reservoir 98 may be simply left in place while the device and nasal cavity are purged with the air or gas.

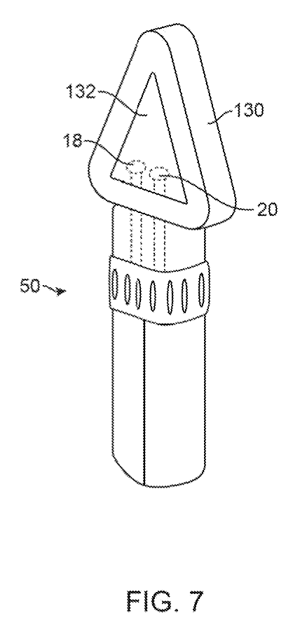

In yet another variation, any of the devices described herein may include a mask 130 optionally attached to the assembly 50, as shown in the perspective view of FIG. 7. Mask 130 may be positioned temporarily upon the user's face in proximity to their nose during a treatment to form a seal against the subject's nose and face to capture any fluid leakage which may occur from the nostril-port interface. Mask 130 may define a receiving channel 132 for positioning the subject's nose therein and may be comprised of a conforming material, such as silicone, to comfortably seal about the nose with lumen openings 18, 20 positioned within for sealing with the user's nostrils.

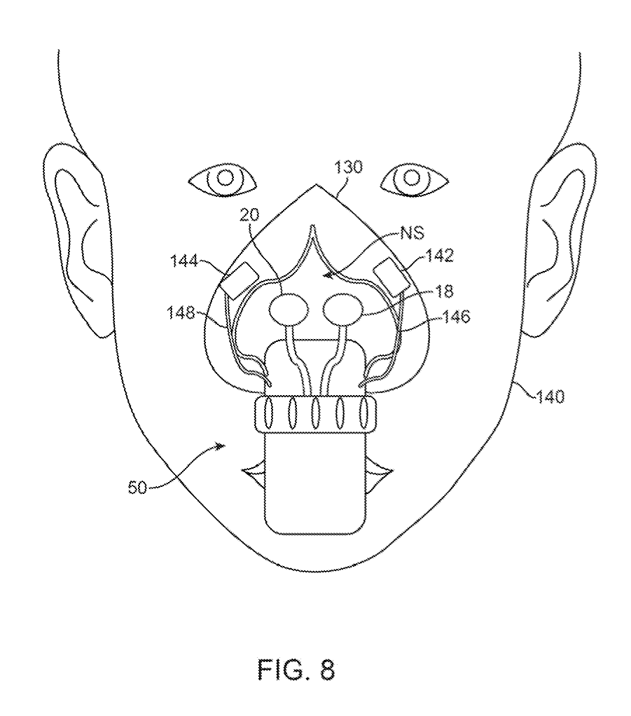

Additionally and/or alternatively in other variations, one or more vibrating elements may be integrated along the mask 130 or assembly 50 which when engaged may vibrate the mask or a portion thereof to transmit vibrations to the underlying tissue or bones, such as the cheek bones, of the subject. These transmitted vibrations may be imparted to disturb any fluids which may be contained within the nasal cavity to cause any debris, such as hardened or thickened mucous, to dislodge from the sinus walls and to mix with the circulating irrigation fluid for flushing out of the nostril and into the capture reservoir. FIG. 8 illustrates a front view of one variation where mask 130 may have the one or more vibrating elements 142, 144 integrated along the mask 130 such that when mask 130 is pressed against the user 140 to form a temporary seal, the vibrating elements 142, 144 may be pressed against the skin and underlying bone, such as the cheekbones, to cause the underlying skull and nasal cavity to vibrate. Vibration of the elements 142, 144 may be achieved via any number of vibrational mechanisms, e.g., a motor may be coupled to drive an eccentrically positioned mass or elements 142, 144 may comprise a piezoelectric mass system electrically coupled via respective electrical connections 146, 148 to a power supply integrated in the assembly 50.

In other variations, additionally and/or alternatively, a vibrational mechanism may be used to directly transmit vibrations through the irrigation fluid being circulated through the nasal cavity. Such vibrations can comprise a high frequency vibration such as ultrasonic vibrations or even low frequency vibrations, e.g., at a frequency of less than 1000 Hz, to cause the mucous to break down and drain more easily with the circulating irrigation fluid.

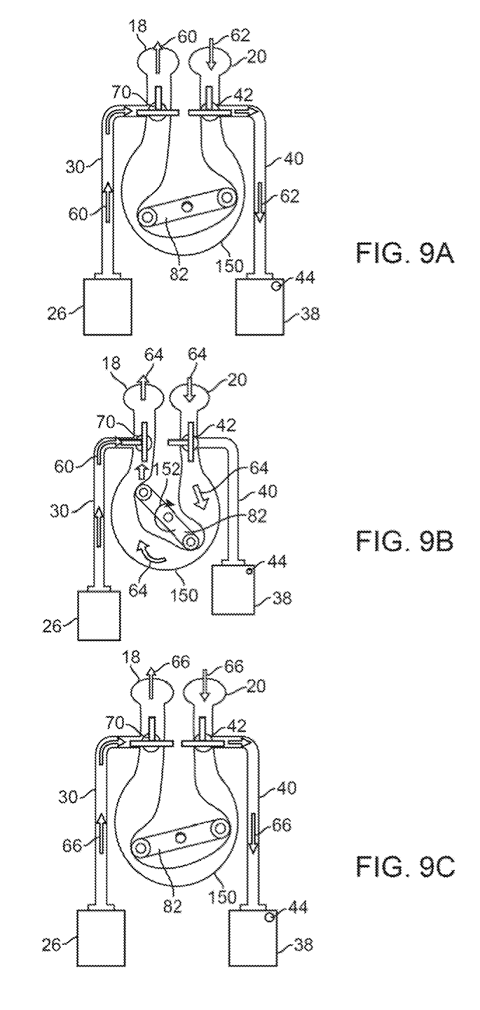

In yet another variation, an irrigation system which may be used for a staged treatment is shown in FIGS. 9A to 9C to illustrate a system utilizing a pulsatile system with a reversible motor-driven peristaltic pump. The peristaltic action that drives the fluid through the device may be completely reversible by reversing the rotation of the motor. Because the peristaltic action of the pump itself results in a pulsatile flow, a flow rate of this pulsatile flow can be controlled by the speed of the rotation of the motor.

Generally, while conventional devices simply flow the irrigation fluid through the nasal cavity, the system herein may be utilized to flow the irrigation fluid through the nasal cavity in a disruptive manner. That is, a pulsatile or vibrational fluid flow may be delivered into and through the nasal cavity in either a rhythmic or synchronized manner or alternatively in a chaotic or turbulent flow pattern where one or more flow parameters, e.g., rate, volume, pressure, direction, etc., can be varied for a given volume of fluid. Such disruptive flow may facilitate penetration of the irrigation fluid through the nasal cavity as well as with removal of any debris or mucous. Moreover, such flow parameters may be controlled via a controller such as a microprocessor or through other mechanical mechanisms, e.g., offset or asymmetric aligned rollers in a peristaltic pumping device, as further described herein.

As illustrated in FIG. 9A, valves 70 and 42 may be set to direct the flow of irrigation fluid 60 from fluid reservoir 26, through the nasal cavity, and into capture reservoir 38. Pump 82, shown as a reversible peristaltic pump, may be initially turned off during this initial purge. The valves 70 and 42 may then be set to direct the fluid 64 in a recirculating pattern through the fluid re-circulation channel 150 and through the nasal cavity by actuating pump 82 in a first direction of rotation 152, as shown in FIG. 9B. With the recirculation cycle completed, valves 70 and 42 may be reset to re-direct the flow from fluid reservoir 26, through the nasal cavity, and back into capture reservoir 38 to allow purging air or gas 66 to cycle through the system and nasal cavity, as shown in FIG. 9C. Pump 82 may be shut off during this treatment phase.

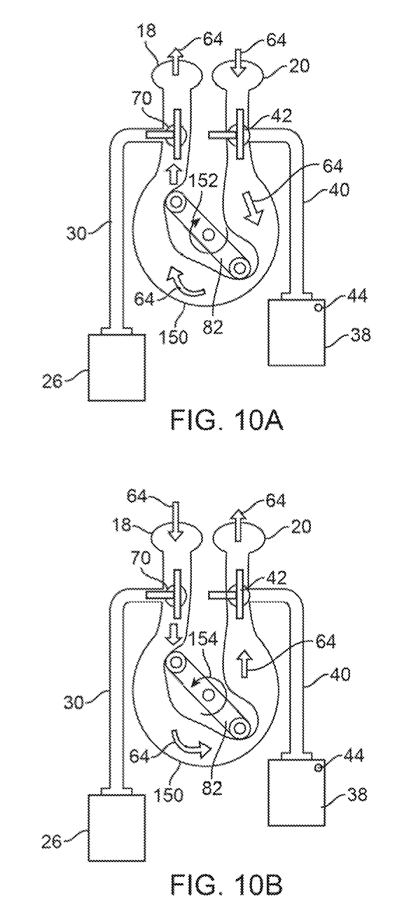

As previously mentioned, because the peristaltic pump 82 is completely reversible, fluid flow during the recirculation phase may be urged to flow through the device and nasal cavity in a first direction, as indicated by the first direction of rotation 152 of the pump 82, as shown in FIG. 10A. During recirculation (or during any phase of the treatment), pump 82 may be optionally reversed to cycle in an opposite second direction of rotation 154, as shown in FIG. 10B, to reverse the flow direction through the device and nasal cavity. This optional reversal of the flow direction may be done periodically or sequentially during treatment to further pulse the irrigation fluid in order to loosen any debris within the nasal cavity as well as to ensure thorough irrigation and/or rinsing of the nasal cavity.

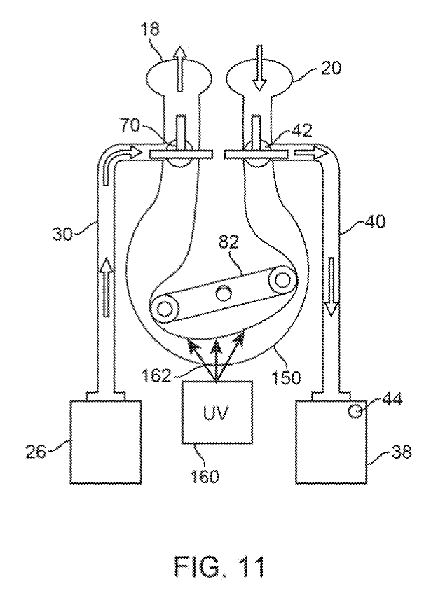

In yet another variation, a high-energy radiation source (e.g., having wavelengths between 185 nm to 245 nm such as an ultraviolet light source 160) may be optionally integrated into the irrigation assembly, as shown in FIG. 11, and positioned within the housing in proximity to fluid channel 150 such that the ultraviolet light source 160 may irradiate 162 the adjacent fluid channel 150 and the irrigation fluid flowing therethrough. The irradiating ultraviolet light 162 may be set at a frequency to kill any microorganisms which may be present in the circulated fluid during the recirculation phase to reduce the chance of spreading infection from one site to another within the nasal cavity. The ultraviolet light source 160 may be directed toward the fluid channel 150 and the housing that may encase the ultraviolet light source 160, fluid channel 150, and peristaltic pump 82 may be made of an ultraviolet resistant material to eliminate the risk of ultraviolet exposure to the subject or others in the area during use.

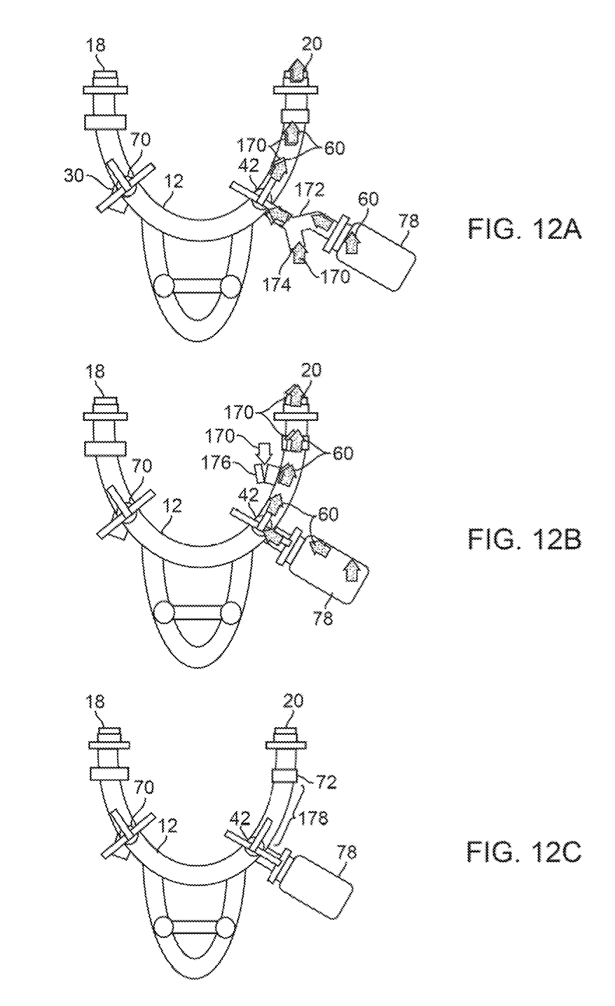

Additionally and/or alternatively, any of the variations described herein may further comprise an optional feature to mix the irrigation fluid with air or a gas introduced into the fluid flow to form discrete volumes for flushing through the device and nasal cavity. This mixture of air (or gas) with irrigation fluid may allow for better disruption of debris within the nasal cavity. When the irrigation fluid 60 is drawn from the reservoir 78, a connector 172 such as a Y-connector connected to the reservoir 78 and also having an opening 174 to ambient air may draw both the fluid 60 and air 170 simultaneously such that they mix while being drawn through fluid channel 12 and into the nasal cavity, as shown in the variation of FIG. 12A.

FIG. 12B shows another variation where a flapper valve 176 may be integrated along fluid channel 12 to allow for mixing between air 170 and the irrigation fluid. As the irrigation fluid 60 is drawn from reservoir 78 and into fluid channel 12, air 170 may also be drawn into fluid channel 12 through flapper valve 176 which may open and close at a specified rate that may be timed with the peristaltic flow. Additionally, flapper valve 176 may be actuated by the peristaltic pump motor. This mixture of air 170 and irrigation fluid within the channel 12 with the peristaltic pump providing the flow may cause discrete boluses of air and fluid to be forced through the system to potentially allow for better disruption of debris within the nasal cavity.

In yet another variation, FIG. 12C illustrates an example utilizing an inline relief valve 72 which may be configured to allow pressure to build within fluid channel 12 along a region of pressure increase 178. When the peristaltic pump is engaged, it may draw the irrigation fluid into the region 178. As pressure builds along fluid channel 12, the pressure may reach a predetermined critical level forcing valve 72 to open. Once valve 72 opens, pressurized fluid may be released into the system and nasal cavity until the pressure along region 178 drops and the pressure building cycle repeats.

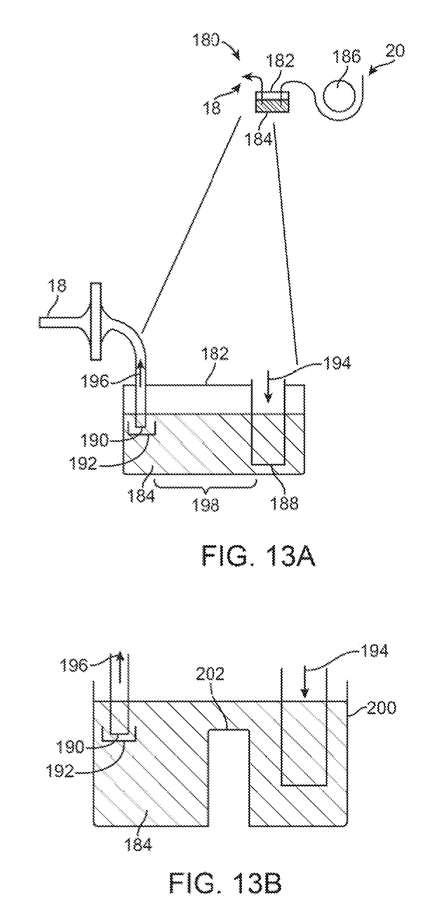

Yet another variation is shown schematically in FIG. 13A for a trap assembly which may be optionally utilized with the systems herein. In this variation, a trap assembly 180 may be integrated along the fluid channel 12 to facilitate adjustment of the degree of pulsation as well as the overall flow rate. A trap 182, e.g., a simple fluid trap, gravity-fed drain, vortex or cyclone trap, etc. may comprise a dampening fluid reservoir 184 which is vacuum sealed and contains an inlet opening 188 spaced apart by a separation distance 198 from an outlet opening 190 leading to, e.g., lumen opening 18.

As pump 186 urges the recirculated irrigation fluid 194 into trap 182, the fluid may exit opening 188 and deposit any debris within reservoir 184 along the separation distance 198. An additional filter 192 may be positioned adjacent to opening 190 to further prevent any debris from entering the fluid channel as the fluid 196 exits trap 182 for entry into the nasal cavity. Trap assembly 180 may also be utilized to dampen the pulsation of the irrigation fluid, if so desired.

Another variation is illustrated schematically in the side view of FIG. 13B, which shows a dampening fluid reservoir 200 having a barrier or projection 202 positioned between the outlet and inlet within the trap. The use of a barrier or projection 202 may further dampen any pulsatile forces as well as inhibit any debris from entering the fluid channel.

Turning now to the filter, repeated passes of fluid during a recirulatory flow through the nasal cavity may cause dislodged debris and mucus to accumulate in the filter. If enough material is accumulated, the filter may eventually become clogged or obstructed to the point that the filter itself disrupts or obstructs the circulation of the irrigation fluid through the device. Accordingly, various mechanisms may be optionally implemented to prevent or inhibit filter obstruction during use of the device.

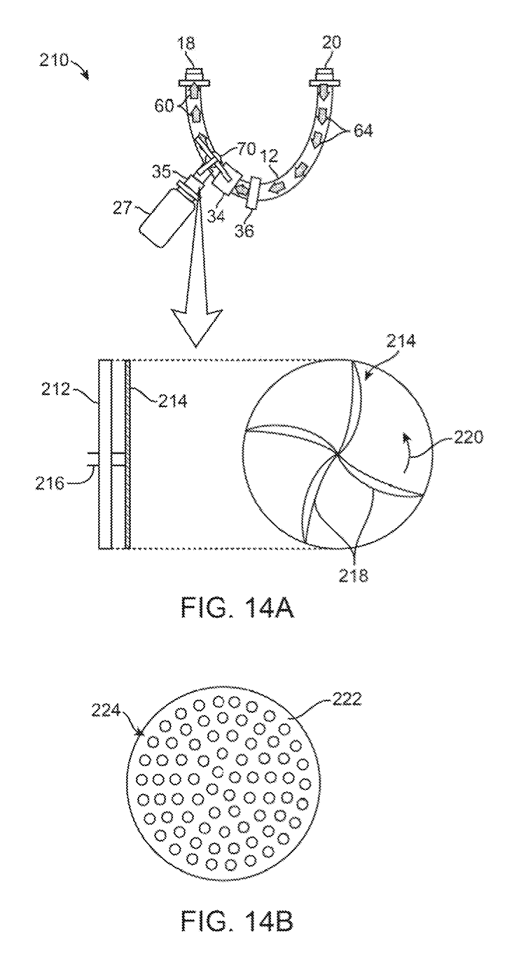

One example is shown in the side and front views of FIG. 14A, which show detail views of a filtering assembly which may be incorporated into any one or all of the filters, such as the removable filter 35 optionally integrated with the detachable reservoir 27, from the device 210 as described herein. In this example, filter element 212 (such as a pad or depth filter) may have a distributor element 214 positioned proximal (i.e., upstream relative to the irrigation fluid flow) to the filter element 212. Distributor element 214 may be positioned relative to filter element 212 via a shaft or support 216 upon which distributor element 214 may rotate and distributor element 214 may further define one or more blades or members 218 which extend from support 216 so as to extend over a diameter of filter element 212. As filter element 212 becomes obstructed by debris and/or mucus, distributor element 214 may be actuated to rotate, e.g., in a first direction 220 about support 216 or even in an oscillating motion, to scrape or disrupt the debris collected on the filter element 212 to allow for the fluid to pass through the filter unobstructed. In other variations, distributor element 222 may be configured as a mesh or sieve defining a plurality of openings 224 to allow for fluid flow therethrough, as shown in FIG. 14B. As the circulation of irrigation fluid occurs, the distributor element may be actuated on an as-needed basis or periodically. Alternatively, the distributor element may be actuated to operate on a continuous basis when the irrigation fluid is set to flow.

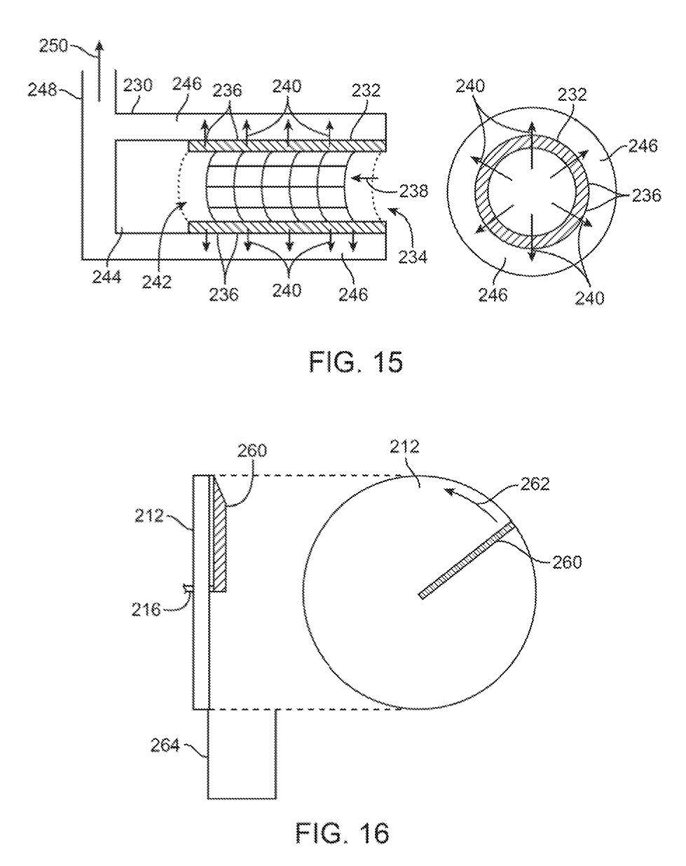

Another variation is shown in the cross-sectional side and end views of a cartridge assembly in FIG. 15. In this variation, a pre-filter or mesh assembly 232 may be positioned within a housing 230 such that the circulated fluid 238 enters into a first opening 234. Housing 230 may be optionally integrated with the reservoir as a singular cartridge, as previously described. The pre-filter assembly 232 may be formed to have a plurality of meshed pores or openings 236 and be configured in a number of shapes, such as a cylindrical shape. A second opening 242 of pre-filter assembly 232 may connect to a collection trap 244 within which the debris and/or mucous may be collected while the fluid is forced to enter first opening 234 and pass through the pores or openings 236, as shown by the filtered fluid 240, while the debris is inhibited from passing through and is collected within collection trap 244. The filtered fluid 240 may then pass into a second channel 246 for collection into fluid channel 248 to continue its recirculatory flow 250 to the nasal cavity. Once the irrigation and/or rinsing procedure is completed, the housing 230 and pre-filter assembly 232 may be removed along with the reservoir containing the collected fluid.

In yet another variation, the distributor element 260 may be configured as a singular blade or member which may be rotated, e.g., in a first direction 262 or in an oscillatory motion, over the filter element 212 to disrupt any collected debris, which may be collected in a collection trap 264. As above; distributor element 260 may be actuated on an as-needed, periodic, or continual basis to maintain the irrigation flow through the filter element 212.

The applications of the devices and methods discussed above are not limited to nasal irrigation and/or rinsing but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A nasal irrigation system, comprising:
a fluid channel in communication with a first lumen opening sized for communication through a first nostril and with a second lumen opening sized for communication through a second nostril of a subject;
a fluid reservoir in communication with the fluid channel;
a capture reservoir in communication with the fluid channel; and,
a valve positionable to a first setting which directs fluid into the capture reservoir and to a second setting which circulates fluid through the fluid channel.

2. The system of claim 1 wherein the fluid channel comprises a tubing member.

3. The system of claim 1 further comprising a first sealing member sized for sealing the first lumen opening to the first nostril.

4. The system of claim 3 further comprising a second sealing member sized for sealing the second lumen opening to the second nostril.

5. The system of claim 1 further comprising a uni-directional valve located along the fluid channel such that fluid flow is restricted to a single direction through the fluid channel.

6. The system of claim 1 further comprising a filter located along the fluid channel.

7. The system of claim 6 further comprising a pre-filtering mechanism in contact with the filter for maintaining uninhibited flow through the filter.

8. The system of claim 1 further comprising a bypass lumen in fluid communication with the fluid channel.

9. The system of claim 1 further comprising an actuation mechanism in communication with the fluid reservoir.

10. The system of claim 9 wherein the actuation mechanism comprises a pump.

11. The system of claim 10 wherein the pump comprises a peristaltic pump.

12. The system of claim 11 wherein the peristaltic pump is reversible such that fluid flow through the fluid channel is reversible.

13. The system of claim 1 further comprising a selector control which is operable between a first position which allows for unidirectional flow through the fluid channel between the fluid reservoir and the capture reservoir and a second position which allows for circulatory flow through the fluid channel.

14. The system of claim 13 wherein the selector control is further operable to a third position which prohibits flow between the fluid reservoir and the capture reservoir.

15. The system of claim 1 further comprising a mask which is conformable about a nose and/or face of the subject.

16. The system of claim 14 further comprising one or more vibrational elements positioned along the mask.

17. The system of claim 1 further comprising a radiation energy light source in communication with the fluid channel.

18. The system of claim 1 further comprising a Y-valve coupled to the fluid reservoir and to an air source.

19. The system of claim 1 further comprising a flapper valve coupled to the fluid channel.

20. The system of claim 1 further comprising a trap assembly in fluid communication with the fluid channel.

21. The system of claim 1 wherein the fluid reservoir is sized to hold between 3 and 20 cc of fluid.

22. The system of claim 1 further comprising a volume of saline fluid contained within the fluid reservoir.

23. The system of claim 22 further comprising one or more agents mixed with the volume of saline fluid.

24. The system of claim 1 wherein the capture reservoir defines a vent opening.

25. A nasal irrigation system, comprising:
a fluid channel in communication with a first lumen opening sized for communication through a first nostril and with a second lumen opening sized for communication through a second nostril of a subject;
a fluid reservoir in communication with the fluid channel;
a capture reservoir in communication with the fluid channel; and,
a selector control which is operable between a first position which allows for unidirectional flow through the fluid channel between the fluid reservoir and the capture reservoir and a second position which allows for circulatory flow through the fluid channel.

26. The system of claim 25 wherein the selector control is further operable to a third position which prohibits flow between the fluid reservoir and the capture reservoir.

27. The system of claim 25 further comprising a uni-directional valve located along the fluid channel such that fluid flow is restricted to a single direction through the fluid channel.

28. The system of claim 25 further comprising a filter located along the fluid channel.

29. The system of claim 28 further comprising a pre-filtering mechanism in contact with the filter for maintaining uninhibited flow through the filter.

30. The system of claim 25 further comprising a bypass lumen in fluid communication with the fluid channel.

31. The system of claim 25 further comprising an actuation mechanism in communication with the fluid reservoir.

32. The system of claim 31 wherein the actuation mechanism comprises a pump.

33. The system of claim 25 further comprising a mask which is conformable about a nose and/or face of the subject.

34. The system of claim 33 further comprising one or more vibrational elements positioned along the mask.

35. The system of claim 25 further comprising a radiation energy light source in communication with the fluid channel.

36. The system of claim 25 further comprising a Y-valve coupled to the fluid reservoir and to an air source.

37. The system of claim 25 further comprising a flapper valve coupled to the fluid channel.

38. The system of claim 25 further comprising a trap assembly in fluid communication with the fluid channel.

39. The system of claim 25 wherein the fluid reservoir is sized to hold between 3 and 20 cc of fluid.

40. The system of claim 25 further comprising a volume of saline fluid contained within the fluid reservoir.

41. The system of claim 40 further comprising one or more agents mixed with the volume of saline fluid.

42. The system of claim 25 wherein the capture reservoir defines a vent opening.

43. A method of irrigating a nasal cavity of a subject, comprising:
introducing a first volume of irrigation fluid from a fluid reservoir into a first nostril such that the first volume passes through the nasal cavity and exits a second nostril into a capture reservoir;
introducing a second volume of irrigation fluid from the fluid reservoir and recirculating the second volume through the nasal cavity; and
purging the second volume from the nasal cavity into the capture reservoir.

44. The method of claim 43 wherein introducing a first volume comprises introducing 3 to 10 cc of the irrigation fluid into the first nostril.

45. The method of claim 43 wherein introducing a first volume comprises introducing saline fluid into the first nostril.

46. The method of claim 45 further comprising introducing one or more agents mixed with the saline fluid into the first nostril.

47. The method of claim 43 wherein introducing a second volume comprises introducing 10 to 20 cc of the irrigation fluid into the first nostril.

48. The method of claim 43 wherein introducing a second volume comprises recirculating the second volume through the nasal cavity for at least two passes.

49. The method of claim 43 wherein introducing a second volume further comprises irradiating the irrigation fluid while recirculating.

50. The method of claim 43 wherein introducing a second volume further comprises filtering the irrigation fluid while recirculating.

51. The method of claim 50 further comprising clearing a filter of debris and/or mucus while recirculating the irrigation fluid.

52. The method of claim 43 further comprising transmitting vibrations to the skull such that debris and/or mucus within the nasal cavity is loosened.

53. The method of claim 43 wherein introducing a second volume comprises urging the irrigation fluid through the nasal cavity via a peristaltic pump.

54. The method of claim 43 wherein introducing a second volume comprises passing the second volume into the first nostril and out of the second nostril.

55. The method of claim 54 further comprising reversing a flow direction of the irrigation fluid such that the second volume passes into the second nostril and out of the first nostril.

56. The method of claim 43 wherein introducing a second volume further comprises restricting the irrigation fluid to flow in a single direction.

57. The method of claim 43 wherein introducing a second volume further comprises selecting a control from a first position which allows for unidirectional flow between the fluid reservoir and the capture reservoir to a second position which allows for circulatory flow through the nasal cavity.

58. The method of claim 43 wherein introducing a second volume further comprises introducing air or gas into the irrigation fluid such that the irrigation fluid is pulsed.

59. A nasal irrigation system, comprising:
a fluid channel in communication with a first lumen opening sized for communication through a first nostril and with a second lumen opening sized for communication through a second nostril of a subject;
a reservoir in communication with the fluid channel and with first and second lumen openings; and,
a valve positionable to a first setting which directs fluid from the reservoir and to a second setting which redirects fluid circulated through the fluid channel back into the reservoir.

60. The system of claim 59 wherein the fluid channel comprises a tubing member.

61. The system of claim 59 further comprising a first sealing member sized for sealing the first lumen opening to the first nostril.

62. The system of claim 61 further comprising a second sealing member sized for sealing the second lumen opening to the second nostril.

63. The system of claim 59 further comprising a uni-directional valve located along the fluid channel such that fluid flow is restricted to a single direction through the fluid channel.

64. The system of claim 59 further comprising a filter integrated with the reservoir.

65. The system of claim 64 further comprising a pre-filtering mechanism in contact with the filter for maintaining uninhibited flow through the filter.

66. The system of claim 59 further comprising an actuation mechanism in communication with the reservoir.

67. The system of claim 66 wherein the actuation mechanism comprises a pump.

68. The system of claim 59 further comprising a mask which is conformable about a nose and/or face of the subject.

69. The system of claim 59 further comprising one or more vibrational elements positioned along the mask.

70. The system of claim 59 further comprising a radiation energy light source in communication with the fluid channel.

71. The system of claim 59 wherein the fluid reservoir is sized to hold less than 20 cc of fluid.

72. The system of claim 59 wherein the reservoir is removably connected to the fluid channel.

73. A method of irrigating a nasal cavity of a subject, comprising:
introducing a volume of irrigation fluid from a reservoir into a first nostril such that the volume passes through the nasal cavity and exits a second nostril into a fluid channel;
recirculating the volume through the fluid channel and nasal cavity; and
purging the volume back into the reservoir.

74. The method of claim 73 wherein introducing a volume comprises introducing less than 20 cc of the irrigation fluid into the first nostril.

75. The method of claim 73 wherein introducing a volume comprises introducing saline fluid into the first nostril.

76. The method of claim 73 further comprising introducing one or more agents mixed with the irrigation fluid into the first nostril.

77. The method of claim 73 recirculating the volume comprises circulating the volume through the nasal cavity for at least two passes.

78. The method of claim 73 recirculating the volume comprises irradiating the irrigation fluid while recirculating.

79. The method of claim 73 recirculating the volume comprises filtering the irrigation fluid while recirculating.

80. The method of claim 79 further comprising clearing a filter of debris and/or mucus while recirculating the irrigation fluid.

81. The method of claim 73 further comprising transmitting vibrations to the skull such that debris and/or mucus within the nasal cavity is loosened.

82. The method of claim 73 recirculating the volume comprises restricting the irrigation fluid to flow in a single direction.

83. The method of claim 73 wherein purging the volume comprises actuating a valve to direct the irrigation fluid back into the reservoir.

84. The method of claim 73 further comprising detaching the reservoir from the fluid channel.
